(19) United States
(12) Patent Application Publication
(10) Pub. No.: US 2016/0228213 A1
(43) Pub. Date: Aug. 11, 2016

(54) ORTHODONTIC TREATMENTS

(71) Applicants: Marcus Alexander TOD, Brisbane, Queensland (AU); Kent Stanley FARMER, Brisbane, Queensland (AU)

(72) Inventors: Marcus Alexander TOD, Brisbane (AU); Kent Stanley FARMER, Brisbane (AU)

(21) Appl. No.: 15/024,206

(22) PCT Filed: Oct. 15, 2014
(86) PCT No.: PCT/AU2014/050287

§ 371 Date: Mar. 23, 2016

(30) Foreign Application Priority Data
Oct. 15, 2013 (AU) 2013903967

(51) Int. Cl. A61C 7/00; A61B 1/00; G06F 19/00; A61B 1/24

(52) U.S. Cl. CPC A61C 7/002; A61B 1/24; A61B 1/00009; A61B 1/0005; G06F 19/345; G06F 19/321

(57) ABSTRACT

The present invention relates to a method for determining suitability of an orthodontic treatment. The method is implemented with at least one computational device and involves posing one or more queries relating to a potential patient. One or more responses to the queries are received and the suitability of the orthodontic treatment is determined based upon the received responses. Advantageously, the potential patient may conveniently perform a self-determination relating to the suitability of the orthodontic treatment prior to arranging a formal assessment with an orthodontist.

### TECHNICAL FIELD

The present invention generally relates to orthodontic treatments such as braces.

### BACKGROUND

The reference to any prior art in this specification is not, and should not be taken as an acknowledgement or any form of suggestion that the prior art forms part of the common general knowledge.

Orthodontics is a specialty of dentistry that is concerned with the study and treatment of malocclusions (improper bites), which may be a result of tooth irregularity or disproportionate jaw relationships.

A commonly used orthodontic treatment is dental braces whereby metal wires are inserted into orthodontic brackets, which can be made from stainless steel or a more aesthetic ceramic material. The wires interact with the brackets to move teeth into the desired positions. Other treatment methods may include aligners or retainers that move teeth.

The Applicant has noticed that some potential patients are inhibited from consulting with an orthodontist for an initial assessment. Furthermore, patients in remote locations are often not inclined to arrange the initial assessment for fear that the time and cost associated with arranging the assessment would be for nothing in the event that the treatment cost is beyond their means.

Embodiments of the present invention provide a means for quelling such assessment inhibitions of potential orthodontist patients.

### SUMMARY OF THE INVENTION

According to one aspect of the present invention, there is provided a method for determining suitability of an orthodontic treatment, the method including the steps of at least one computational device:

posing one or more queries relating to a potential patient;

receiving one or more responses to the queries; and

determining the suitability of the orthodontic treatment based upon the received responses.

Advantageously, the potential patient may conveniently perform a self determination relating to the suitability of the orthodontic treatment prior to arranging a formal assessment with an orthodontist.

The method may further include the step of capturing image of the patient's teeth. The step of capturing may involve displaying a reference location or orientation (or locating or orientating the teeth in the captured image. The method may further include the step of selecting a tooth of the captured image. The method may further include the step of associating a problem with the selected tooth. The problem may include a tooth sticking out, a crooked tooth or a gap between teeth.

The queries may relate to the type of bite of the patient, individual teeth of the patient. The responses may be selections to which weights are assigned.

The orthodontic treatment may be a reduced treatment, thereby encouraging these with limited means to seek treatment. The suitability may be a percentage determined using the assigned weights. The suitability may be displayed graphically and as being proportional to the simplicity of the patient's case.

The method may further include the step of providing a suitability report. The report may include the percentage. The report may further include a predicted treatment time, cost estimate of the treatment, and an indication of a suggested treatment option.

The queries and responses may relate to personal information. The personal information may relate to one or more of potential patient identity, the age of the potential patient, the time that the potential patient has beer considering treatment, the name and postcode of the potential patient, any concerns of the potential patient, and benefits of the treatment.

The method may further involve capturing further images of the patient's teeth over time. The method may involve enabling sequential display of the images showing any change in the patients teeth. The method may further involve prompting the patient to capture the image. The prompting may occur periodically (e.g. weekly). The method may involve providing a progress report regarding the number of images captured.

The method may further involve providing or determining the duration until an event (e.g. wedding, school formal). The providing or determining may involve entering the date of the event. The method may involve providing a countdown until the event. The method may involve displaying treatment tips at pre-determined times throughout the countdown based upon the remaining duration. The method may involve displaying treatment options to the patient based upon the duration.

According to another aspect of the present invention, there is provided an orthodontic treatment suitability system including at least one computational device and configured to:

pose one or more queries relating to a potential patient;

receive one or more responses to the queries; and

determine suitability of an orthodontic treatment based upon the received responses.

According to another aspect of the present invention, there is provided a method for tracking the progress of a patient undergoing orthodontic treatment, the method including the steps of at least one computational device:

capturing images of the patient's teeth over time; and

sequentially displaying the images showing any change in the patients teeth.

According to another aspect of the present invention; there is provided a method for motivating a patient undergoing orthodontic treatment, the method including the steps of at least one computational device:

providing or determining the duration until an event associated with the patient; and

providing a countdown until the event.

Any of the features described herein can be combined in any combination with any one or more of the other features described herein within the scope of the invention.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the invention in any way. The Detailed Description Will make reference to a number of drawings as follows:

FIG. 1 is a block diagram of an orthodontic treat suitability system in accordance with an embodiment of the present invention;

FIG. 2 is a flowchart of a method for determining suitability of an orthodontic treatment using the system of FIG. 1;

FIG. 3 shows a mobile phone screen displayed when capturing a smile during the method of FIG. 2;

FIG. 4 shows a mobile phone screen displayed when selecting problem teeth during the method of FIG. 2;

FIG. 5 shows a mobile phone screen displayed when selecting problems with individual teeth during the method of FIG. 2;

FIG. 6 shows a mobile phone screen displayed when selecting a bite type during the method of FIG. 2;

FIG. 7 shows a mobile phone screen displaying a generated suitability report, indicating low suitability for a reduced treatment, during the method of FIG. 2;

FIG. 8 shows a mobile phone screen displaying a generated suitability report, indicating high suitability for a reduced treatment, during the method of FIG. 2;

FIG. 9 shows a mobile phone screen displaying a progress report regarding the number of images already captured;

FIG. 10 shows a mobile phone screen for capturing another image;

FIG. 11 shows a mobile phone screen for initiating a countdown to an event; and

FIG. 12 shows a mobile phone screen displaying options and tips leading up to the event of FIG. 11.

### DETAILED DESCRIPTION OF PREFERRED EMBODIMENTS

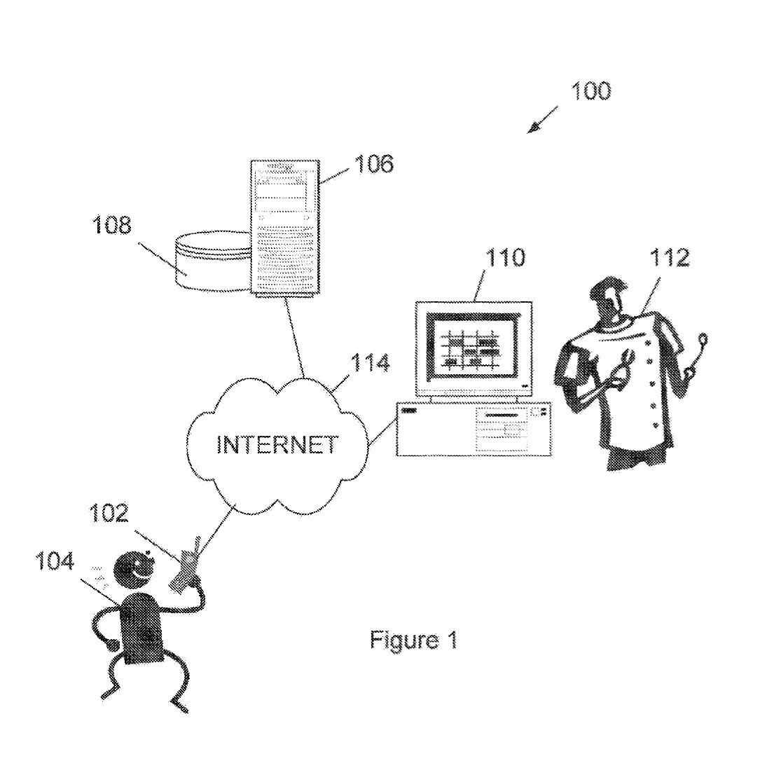

According to an embodiment of the present invention, there is provided an orthodontic treatment suitability system 100 as shown in in FIG. 1. The system 100 includes a mobile phone 102 (i.e. computational device) executing application software (i.e. an App), and equipped with a camera for capturing images of teeth. The smart phone 102 of the system 100 poses one or more queries relating to a potential patient 104 and receives one or more responses to the queries from the patient 104. The system 100 further determines the suitability of an orthodontic treatment based upon the received responses. Advantageously, the potential patient 104 can conveniently perform a self determination relating to the suitability of the orthodontic treatment, prior to arranging a formal assessment with an orthodontist.

The system 100 further includes an administration server 106 hosting a website and from which the App can be downloaded. The server 106 includes a database 108 storing patient records that include, the responses and a report including the determined suitability.

The system 100 further includes an orthodontists computer 110 which enables an orthodontist 112 to remotely access, over the internet 114, the stored records in the database 108.

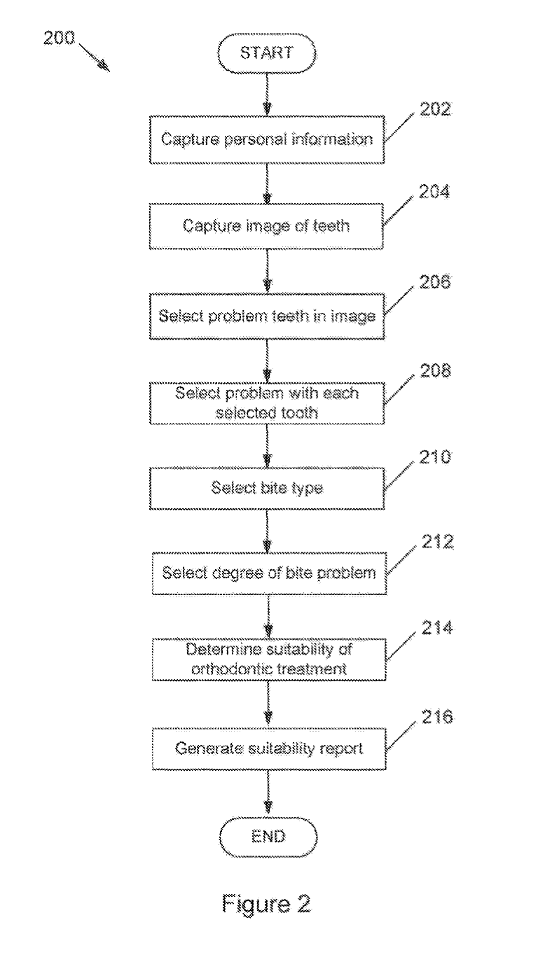

A method 200 for determining suitability of an orthodontic treatment is now described with reference to FIG. 2.

Initially at step 202, the mobile phone 102 of the system 100 executing the App, poses personal information queries relating to a potential patient 104 and receives responses to the queries. The personal information relates to whether or not the treatment is for the phone user or another potential patient 104 (e.g. dependant), the age of the potential patient 104, the time that the potential patient 104 has been considering treatment, and the name and postcode of the potential patient 104.

The personal information also relates to any concerns of the potential patient 104 including whether braces look ugly, whether the patient 104 perceives that treatment is unaffordable, whether braces are applied for too long, whether pain is involved in the treatment, and whether or not there is value in having the treatment (cost verses benefit).

The personal information also relates to perceived benefits of the treatment including more confidence, aesthetics such as a beautiful smile, a new start, and looking good in photos.

Some of the queries may be posed in the form of a checklist or slider bar, and finite responses may be elicited using these tools. Not only do compiled responses stored in the records of database 108 at the administration center 106 provide useful statistical information for the orthodontist 112, but they also enable the orthodontist to better assist individual patients 104 should treatment be sought. Further, the structured personal information queries provide a check to reaffirm that the potential patient 104 is making the right decision in seeking treatment.

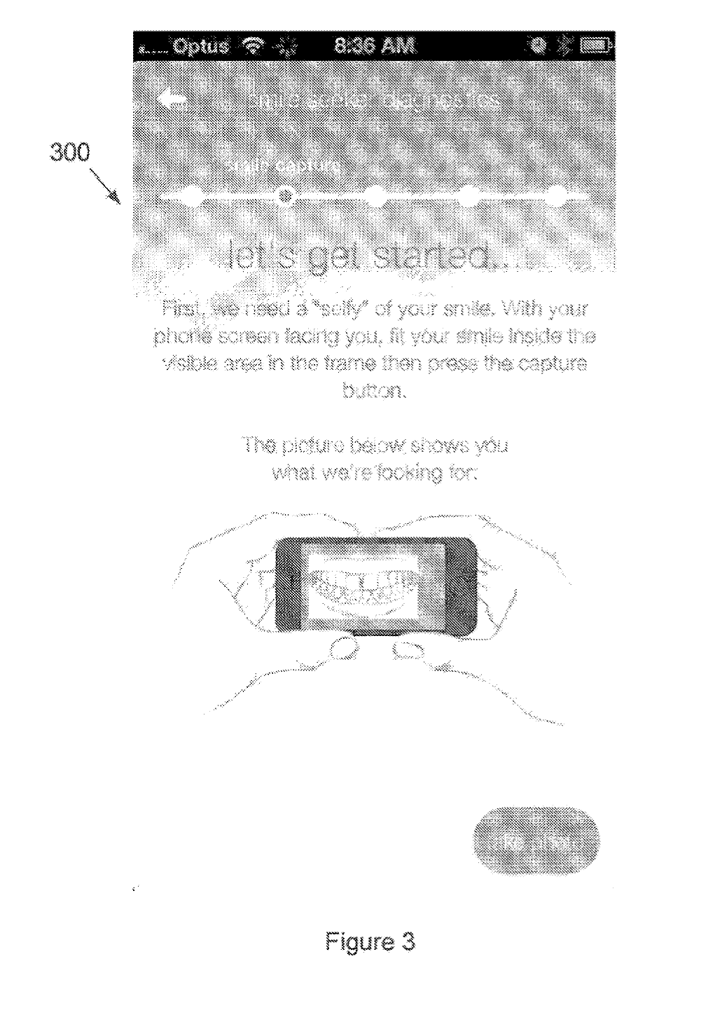

At step 204, the mobile phone camera of the system 100 captures an image of the potential patient's smile. In particular, the App prompts the patient 104 to ea ere the image as shown in the mobile phone screen shot 300 of FIG. 3.

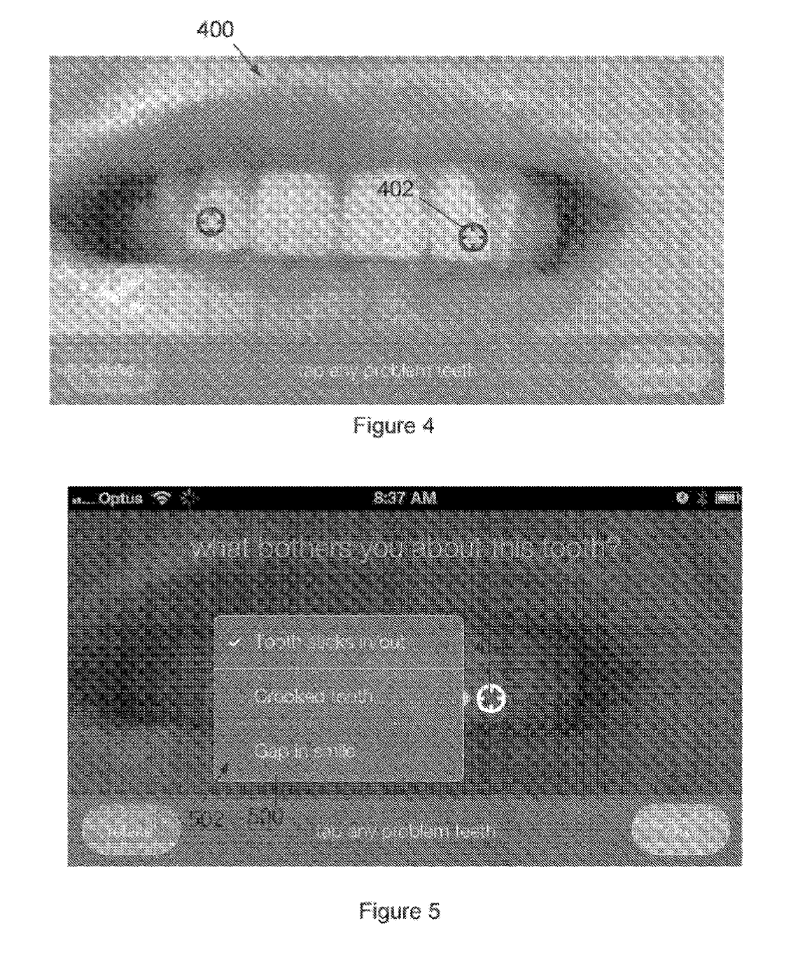

At step 206, the mobile phone 102 of the system 100 queries the patient 104 to select any problem teeth in the captured teeth image 400 as shown in FIG. 4. The phone 102 receives responses as the patient 104 taps on the individual teeth 40, the image 400.

At step 208, the mobile phone 102 receives responses from the patient 104 in the form of selected problems associated with each selected tooth 402. As shown in FIG. 5, the patient 104 selects a problem item 500 from a pick list 502 to identify the problem with each selected tooth 402. The first selected problem tooth (e.g. gap, crooked, angled in, angled out, etc.) is assigned an associated weighted score from Table I below (e.g. first crocked tooth=weighting of 7).

TABLE I

| Individual Teeth | Score |
|---|---|
| Gap | 7 |
| Crooked | 7 |
| Angled In | 9 |
| Angled Out | 9 |

Any additional teeth are assigned a weighting of 5. For example, a first crooked tooth and a second gapped tooth will result in a weighting of 12 (i.e. 7+5)

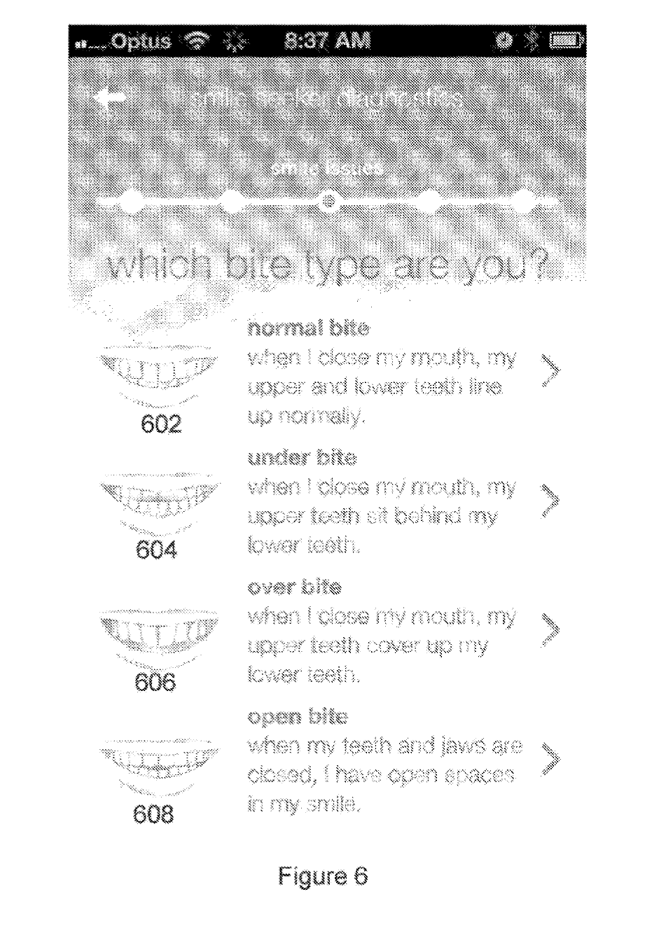

At step 210, the mobile phone 102 queries the type of bite of the patient as shown in FIG. 6. The types of bite include a normal bite 602, under bite 604, over bite 606 and open bite 608. The patient 104 selects a particular type of bite which, for normal bite 602 and open bite $08, is directly assigned a weighted score as shown in Table II below.

TABLE II

| | Bite Type | | Score |
|---|---|---|---|
| Normal | | | 0 |
| | Underbite: | Mild | 26 |
| | | Moderate | 52 |
| | | Severe | 65 |
| | Overbite: | Normal | 26 |
| | | Deep | 52 |
| | | Very Deep | 65 |

| | |
|---|---|
| Openbite | 65 |

At step 212, for an under bite 604 or over bite 608 selection at step 210, the mobile phone 102 queries the degree (e.g. mild, moderate, severe) of the bite condition of the patient 104. The patient 104 selects a degree which is assigned a weighted score as shown in Table II.

At step 214, the mobile phone 102 determines the suitability of the orthodontic treatment based upon the received responses. In particular, the suitability of treatment is a percentage determined using the assigned weights as follows:

Percentage suitability for reduced treatment=sum of weighted individual teeth (table 1, step 208)+weighed bite (table 2, steps 208 & 210)×100 divided by 1.

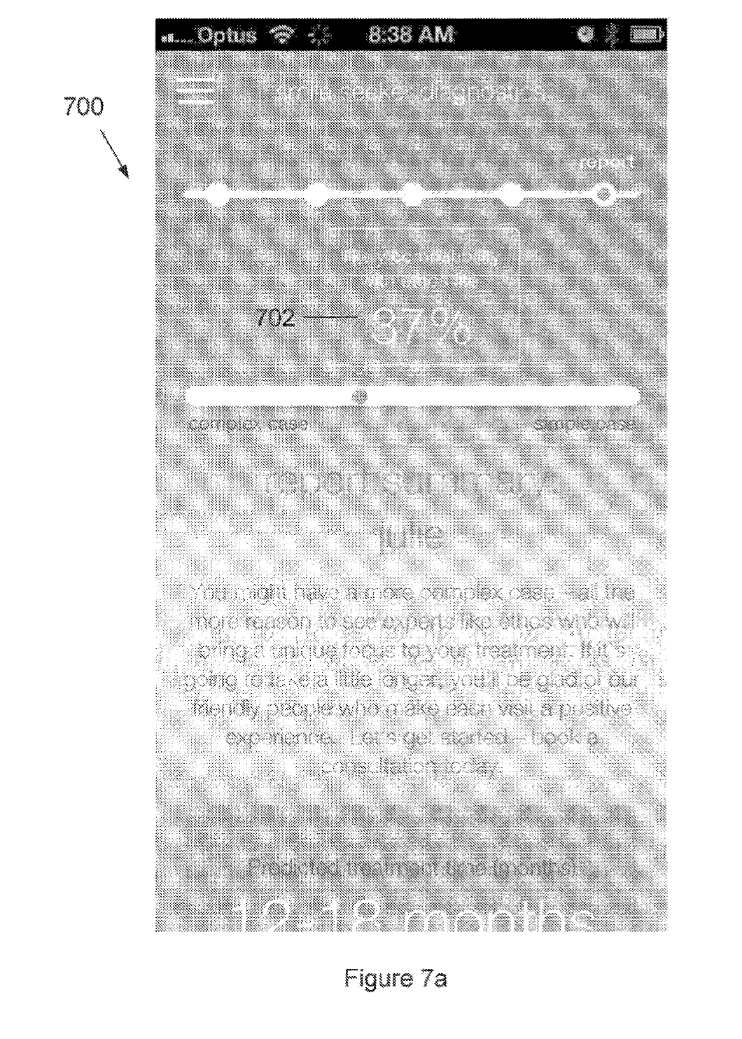

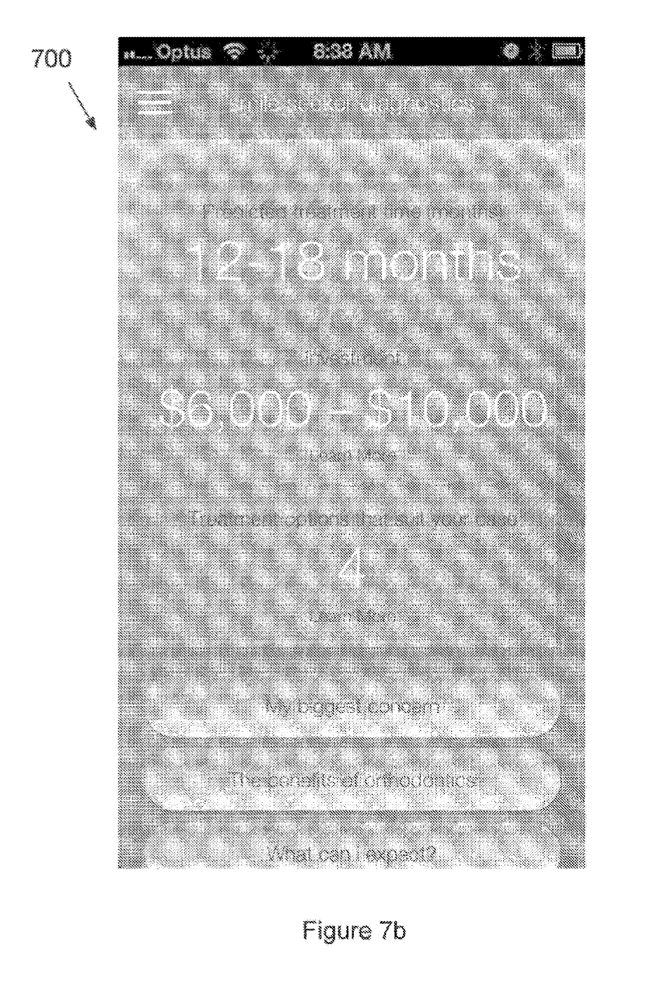

At step 218, the mobile phone 102 provides a suitability report 700 as shown in FIG. 7. The report 700 includes the determined suitability percentage 702 for the treatment which is a reduced treatment. As shown in FIG. 7a, the percentage 702 is also displayed graphically whereby the determined suitability is proportional to the simplicity of the patient's case based upon the responses. As shown in FIG. 7b, the report 700 further includes a predicted treatment time, cost estimate of the treatment, and an indication of a suggested treatment option.

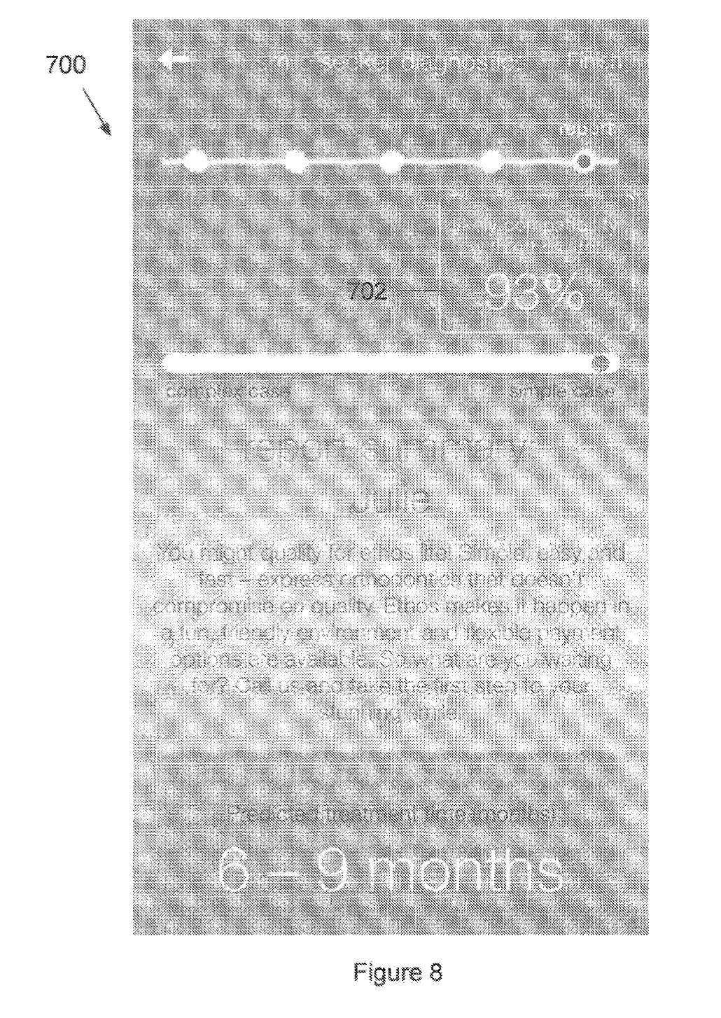

If the percentage 702 is below 50% as shown in FIG. 7a, then a full treatment is recommended. If the percentage 702 is above 50% as shown in FIG. 8, then a reduced orthodontic treatment is suitable thereby encouraging those with limited means to seek treatment.

The report 700 is stored in a client record in the database 108, and can be later accessed over the Internet 114 by the patient 104 or orthodontist 112. At any time, the patient 104 undergoing treatment will be able to log into their account using the App and thereby gain access to the report 700 stored in the client record in the database 108.

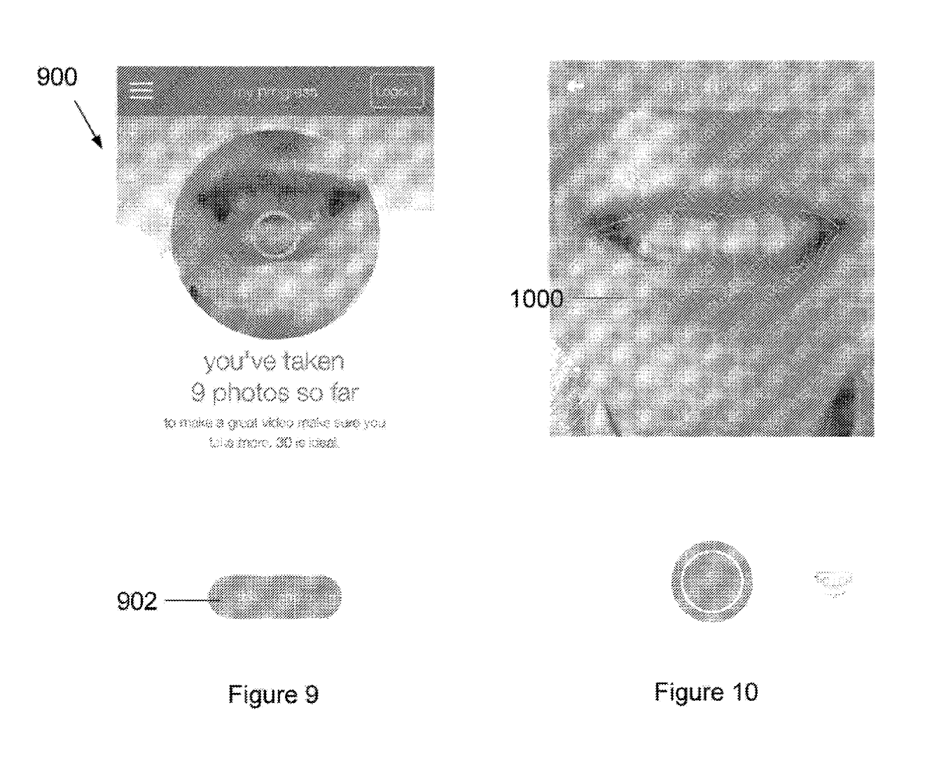

As shown in FIGS. 9 and 10, the patient 104 can also log into their account and capture further images of their teeth over time. The images are stored in the database 108.

Turning to FIG. 9, the patient's smart phone 102 provides a progress report 900 regarding the number of images already captured and stored in the database 108. The smart phone 102 displays the selection button 902 prompting the patient 104 to capture another image. Typically, the administration server 106 periodically (e.g. weekly) prompts the patient via their smartphone 102 (e.g. using SMS or e-mail) to log in and capture a new image.

Turning to FIG. 10, the patient's smart phone 102 displays a reference location and orientation 1000, in the form of silhouetted and superimposed teeth, for locating and orienting the patient's teeth in the captured image. The patient's smart phone 102 can be used to sequentially display the captured images stored in the database 108 in the form of a video thereby showing any change in the patient's teeth over time. The display of this video gives the patient 104 encouragement in seeing the alignment of their teeth during treatment, which may not otherwise be appreciated during a long treatment where changes are somewhat incremental.

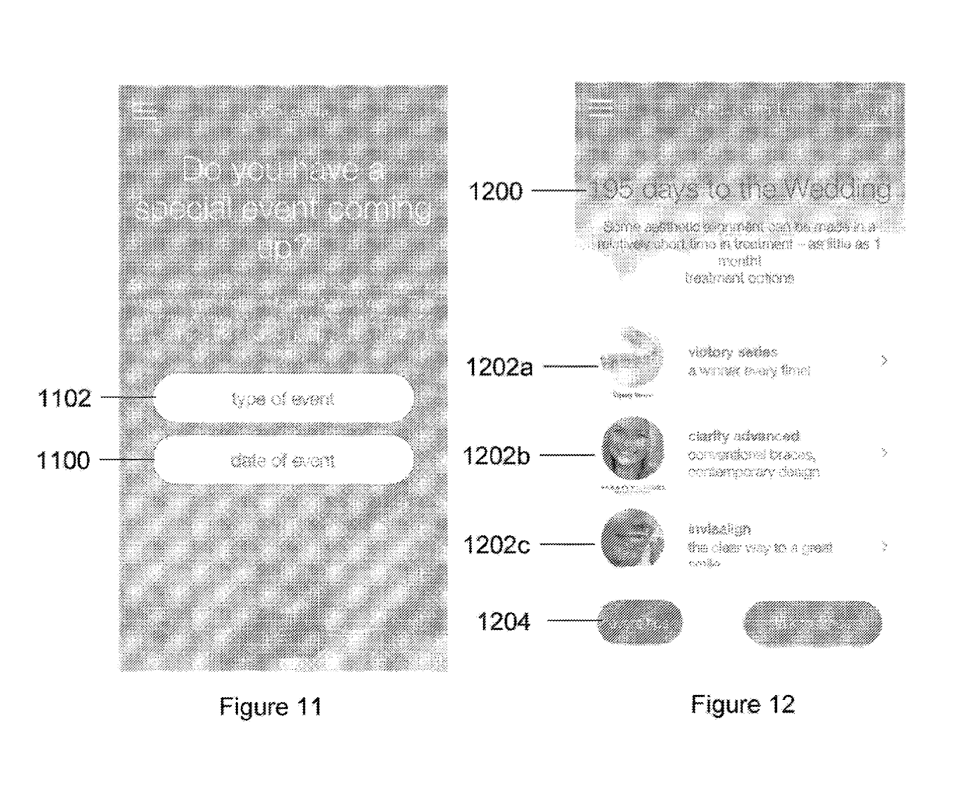

Turning to FIG. 11, the patient 104 can also use the App to enter the date 1100 of an event 1102 (e.g. wedding, school formal, etc), and this information is also stored in the database 108. The duration Until the event is determined and monitored by the administration server 106 which provides a running countdown until the event.

Turning to FIG. 12; the patient's smart phone 102 displays the countdown 1200. The smart phone 102 also displays determined treatment options 1202a, 1202h, 1202c to the patient 104 based upon the duration (or remaining duration) until the event. Furthermore, as the patient 104 logs into their account throughout, treatment, the smart phone 102 also displays treatment tips 1204 (e.g. whitening, oral hygiene, etc.) at pre-determined times (e.g. weekly, monthly, quarterly until event) throughout the countdown based upon the remaining duration. The tips can be sent by e-mail or SMS from the server 106. Although the patient 104 can elect not to receive tips 1204, the tips 1204 help the patient 104 to track their treatment and remain on track for completing their treatment by the event date 1100.

A person skilled in the art will appreciate that many embodiments and variations can be made without departing from the ambit of the present invention.

In compliance with the statute, the invention has been described in language more or less specific to structural or methodical features. It is to be understood that the invention is not limited to specific features shown or described since the means herein described comprises preferred forms of putting the invention into effect.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics May be combined in any suitable manner in one or more combinations.

The claims defining the invention are as follows:

1. A method for determining suitability of an orthodontic treatment, the method including the steps of at least one computational device:
posing one or more queries relating to a potential patient;
receiving one or more responses to the queries; and
determining the suitability of the orthodontic treatment based upon the received responses.

2. The method as claimed in claim 1, further including the step of capturing an image of the patient's teeth.

3. The method as claimed in claim 2, further including the step of selecting a tooth of the captured image.

4. The method as claimed in claim 3, further including the step of associating a problem with the selected tooth.

5. The method as claimed in claim 4, wherein the problem includes a tooth sticking out, a crooked tooth or a gap between teeth.

6. The method as claimed in claim 2, wherein the step of capturing involves displaying a reference location or orientation for locating or orientating the teeth in the captured image.

7. The method as claimed in claim 1, wherein the queries relate to the type of bite of the patient, or individual teeth of the patient.

8. The method as claimed in claim 1, wherein the responses are selections to which weights are assigned.

9. The method as claimed in claim 9, wherein the suitability is a percentage determined using the assigned weights.

10. The method as claimed in claim 1, wherein the suitability is displayed graphically and as being proportional to the simplicity of the patient's case.

11. The method as claimed in claim 1, wherein the orthodontic treatment is a reduced treatment, thereby encouraging those with limited means to seek treatment.

12. The method as claimed in claim 1, further including the step of providing a suitability report.

13. The method as claimed in claim 12, wherein the report includes a suitability percentage.

14. The method as claimed in claim 12, wherein the report includes a predicted treatment time, cost estimate of the treatment, or an indication of a suggested treatment option.

15. The method as claimed in claim 1, wherein the queries and responses relate to personal information.

16. The method as claimed in claim 15, wherein the personal information relates to one or more of potential patient identity, the time that the potential patient has been considering treatment, the name and postcode of the potential patient, any concerns of the potential patient, and benefits of the treatment.

17. The method as claimed in claim 1, further involving capturing further images of the patient's teeth over time and enabling sequential display of the images showing any change in the patient's teeth.

18. The method as claimed in claim 17, further involving prompting the patient to capture the images.

19.-21. (canceled)

22. An orthodontic treatment suitability system including at mast one computational device and configured to:
pose one or more queries relating to a potential patient;
receive one or tore responses to the queries; and
determine suitability of an orthodontic treatment based upon the received responses.

23. A method for tracking the progress of a patient undergoing orthodontic treatment, the method including the steps of at least one computational device:
capturing images of the patient's teeth over time; and
sequentially displaying the images showing any change in the patient's teeth.

24. (canceled)
